(19) United States
(12) Patent Application Publication
(10) Pub. No.: US 2006/0173716 A1
(43) Pub. Date: Aug. 3, 2006

(54) System and method for real time regional feedback

(76) Inventor: Hao Wang, Worcester, MA (US)

Correspondence Address: EPSTEIN BECKER & GREEN, P.C., 1227 25TH STREET, N.W. 7TH FLOOR, WASHINGTON, DC, 20037, US

(21) Appl. No.: 11/341,674

(22) Filed: Jan. 30, 2006

Related U.S. Application Data
(60) Provisional application No. 60/648,390, filed on Feb. 1, 2005.

(51) Int. Cl. G06Q 10/00; A61B 5/00

(52) U.S. Cl. 705/2; 600/300

(57) ABSTRACT

The present invention relates to a regional health data exchange infrastructure comprising a measurement and feedback framework to enable continuous and automatic quality and safety data collection, dissemination, and reporting. The present invention provides near real time monitoring of healthcare environment at the regional and preferably nationally level, across organization boundaries.

### RELATED APPLICATION

This patent application claims priority to U.S. Provisional Application Ser. No. 60/648,390 filed Feb. 1, 2005, which is incorporated herein by reference in its entirety.

### FIELD OF THE INVENTION

The present invention relates to health information technology. Specifically, the present invention relates to real time data collection and analysis in the health care arena.

### BACKGROUND OF THE INVENTION

Over the past two decades a fair amount of research has been conducted to measure patient safety and care quality, which has provided a framework for healthcare measurements and analyses. However, due to the lack of effective data exchange mechanisms, most, if not all, of this research has been confined by limited data sets within an individual organization's boundaries. The measurement is taken by individual organizations, not being shared in real time with their trading partners, or with each other. Usually an individual organization only takes care of one aspect of the patient healthcare, not the entire health need. Therefore, the data collected from the measurement is often not comprehensive and it is hard to see the whole picture from the available data sets. The analysis of these measurement data is also confined to the limited data sets. The result of the analysis is local, not comparable across different organizations. A valid reference benchmark system is hard to establish.

Several government initiatives have promoted standard measurement procedures for patient safety and health care quality. These initiatives have published sets of quality indicators and other indicators to help organizations implement standard measurement. Due to a variation of the implementations of these standard indicators, there is no known benchmark processes today to compare and disseminate individual measurement processes. There are processes of collecting measurement data from different organizations and analyzing them together. However the processes usually take months, and cannot be used in real time to help organizations to make immediate adjustment they need.

The healthcare industry is in need of a distributed data collection, dissemination, and reporting mechanism to provide members of the industry an impartial real time monitoring and measurement system for patient safety and healthcare quality. The healthcare industry is also in need of a region-wide benchmark system to provide the perspectives of progress for improvement of patient safety and care quality.

### SUMMARY OF THE INVENTION

It is an object of the invention to provide, at a system level, a mechanism to collect real-time measurement data for patient safety and healthcare quality.

It is a further object of the invention to implement these standard measurements at a regional level, leveraging the global computer network, across organizational boundaries.

It is a further object of the invention to disseminate these real time measurements and provide standard reports to the participating organizations.

It is a further object of the invention to provide additional value added analysis and reporting services to participating organizations to help them align with the industry standards of patient safety and care quality.

The present invention provides a regional health data exchange infrastructure comprising a measurement framework to enable continuous and automatic quality and safety data collection, dissemination, and reporting. This present invention provides near real time monitoring of healthcare environment at the regional and preferably nationally level, across organization boundaries.

Moreover, through the use of the feedback system disclosed herein, data related to healthcare efficiency can also be tracked and analyzed. The result can be fed to healthcare organizations providing information as to any system deficiency, and thereby facilitating rectification of that deficiency. The community wide quality and safety monitoring can provide a uniform safety and quality standard and baseline to guide organizations through improvements in care delivery and thereby reduce care cost.

By accessing longitudinal consumer health data the present invention also provides for analysis of preventive quality indicators at the regional level. This feedback system also provides a mechanism to mitigate risk and liabilities and reduce the medical malpractice possibilities through improved data collection, documentation, communication, decision support, and standardization.

This present invention also enables the construction of regional adverse drug event reporting systems. Such a reporting and monitoring system can track the community wide adverse drug events, and can also provide mechanisms for near real time intervention by healthcare organizations throughout the entire community.

There has thus been outlined, rather broadly, the more important features of the invention in order that the detailed description thereof that follows may be better understood, and in order that the present contribution to the art may be better appreciated. There are, of course, additional features of the invention that will be described further hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that equivalent constructions insofar as they do not depart from the spirit and scope of the present invention, are included in the present invention.

For a better understanding of the invention, its operating advantages and the specific objects attained by its uses, reference should be had to the accompanying drawings and descriptive matter which illustrate preferred embodiments of the invention.

### BRIEF DESCRIPTION OF THE DRAWINGS

FIG. 1 illustrates the centralized real time monitoring, analysis and reporting system in a local health information infrastructure.

FIG. 2 illustrates real time patient safety and care quality measurement and feedback in a local health information infrastructure

FIG. 3 illustrates real time measurement, monitoring and response in a local health information infrastructure.

FIG. 4 illustrates monitoring and benchmark payors in a local health information infrastructure.

FIG. 5 illustrates real time date monitoring, analysis and response system incorporated within the health care system.

### DETAILED DESCRIPTION OF THE PREFERRED EMBODIMENTS

The present invention comprises a federated data collection and dissemination system that enables regional/national real-time continuous monitoring, measurement, assessment, and feedback on patient safety and healthcare quality measures. Preferably the system is a distributed system with minimal to no central authority. It is a larger system made up by a number of smaller heterogeneous systems, wherein each small system maintains its autonomy. The system is regional and encompasses all members in the healthcare industry. It is able to provide unbiased and cross organizational studies and comparisons. The feedback system comprises a real time data collection system so that patient safety, care quality, as well as operation efficiency can be closely monitored and reported. The system further comprises a benchmarking mechanism that can provide all organizations a reference system to align themselves with the industry's leading performers and strive for better performance.

Architecture

The components of the present invention include but are not limited to messaging capable federated network, medical data receiver, Web service hosting and development module, database(s), federated master person index, business logic module, enterprise reporting module, data warehousing module, and business intelligence module. Network refers to computer network. When multiple computer networks connect together to form a larger network while each individual smaller computer network maintains its autonomy, it becomes what's known as federated network. A messaging capable federated network means such federated network is able to generate, send, transport, and receive messages, which are objects of communication. A medical data receiver is a software component that listens to messages that contains medical data for patients. Often this software component is in the forms of Web services. A Web service hosting and development module is a computer operating system environment that can host Web services and help users develop and manage such Web services. Database is an organized collection of data; it is a collection of records stored in a computer in a systematic way such that users can query for certain information against. Typically a master person index refers to a computer-based database system that facilitates the tracking of patient information by assigning each patient an identifier. A federated master person index means such computer-based index system comprises a number of smaller index systems; each of which maintains its autonomy while collectively the larger federated index system can facilitate the tracking of patient information for a much larger population that a smaller index system can not handle. Business logic module in computer term refers to a computation component that embodies business and operation rules that act on business objects and entities. Enterprise reporting module is a system that produces unified reports which join different data views of an enterprise in one place. The enterprise reporting involves querying data sources with different logical models to produce a human readable report. Data warehousing module is a computer system that houses an enterprise's past transactional and operational information in large amounts, stored in a database designed to favor efficient data analysis and reporting. Business intelligence module refers to a software system that collects and analyzes data to extract valuable business information.

The messaging capable federated network generates, routes, and manages secure messages that can be sent to all members in the system, including government, for public health concerns or other reasons. There are several existing messaging technologies known by those of ordinary skill in the art, which could serve as a messaging engine. Examples of such technology include but are not limited to IBM MQ Series, Microsoft MQ; Java JMS, and the like.

There are several existing business integration technologies known by those of ordinary skill in the art, which could serve as the medical data receptacle and environment for the tool's process orchestration. Examples of such technology include but are not limited to Microsoft BizTalk Server 2004 IBM Business Integration Server; Seebeyond eGate; Novell eXtend, and the like.

There are several existing Web service hosting and development technologies known by those of ordinary skill in the art, which could serve as the Web service hosting environment. Examples of such technology include but are not limited to Microsoft .NET technology including IIS web server and Visual Studio .NET development environment; IBM WebSphere software platform and WebSphere Studio development environment, and the like.

There are several existing database technologies known by those of ordinary skill in the art. Examples of such technology include but are not limited to Microsoft SQL Server 2000, IBM DB2, Oracle 9i, and the like.

There are several existing enterprise reporting technologies known by those of ordinary skill in the art. Examples of such technology include but are not limited to Crystal Report Enterprise Edition, Microsoft SQL Server reporting services, and Active Report from DataDynamics. In a preferred embodiment such enterprise reporting is available to the user via Web services.

There are several existing data warehousing technologies known by those of ordinary skill in the art. Examples of such technology include but are not limited to Oracle data warehouse, Microsoft SQL server, IBM DB2 data warehousing, and Cognos and Brio data warehouse tools.

There are several existing business intelligence tools known by those of ordinary skill in the art. Examples of such tools are provided by companies including but are not limited to Brio, Cognos IBM, Microsoft, and Oracle.

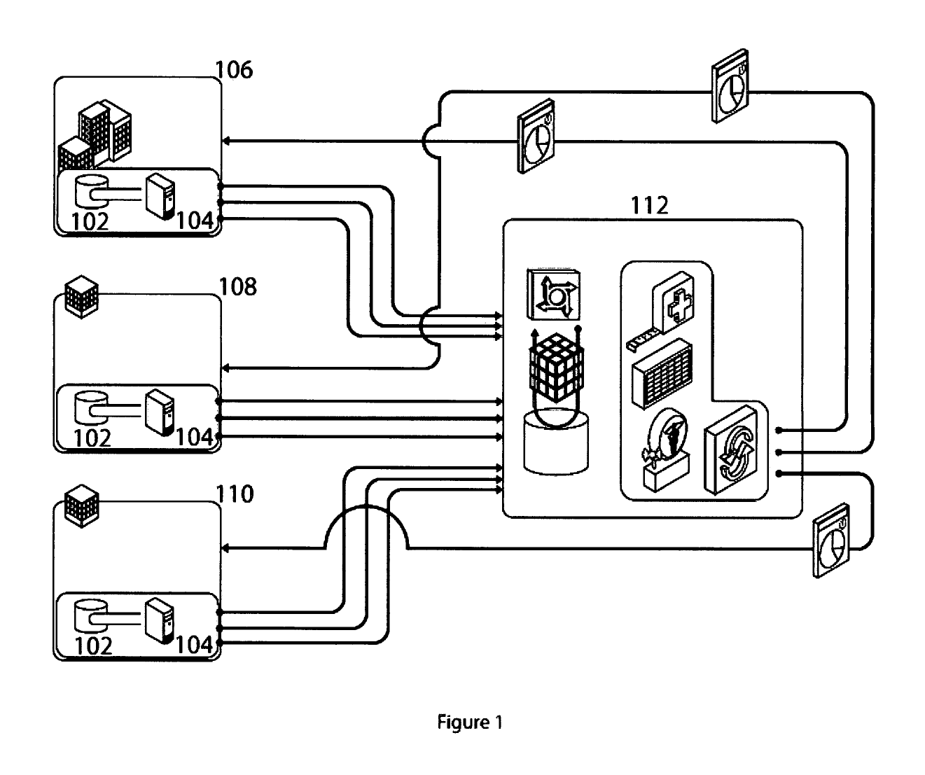

Preferably these modules are provided in a service oriented environment to enable the participating organization in sending data in real time to the central data warehouse. See for example, FIG. 1.

The present invention comprises at least one federated person index that can be used to map people from multiple independent data sources. This federated person index is described in detail in U.S. patent application Ser. No. 11/117,499 entitled “Data Sharing Infrastructure”, which is hereby incorporated by reference in its entirety. At least one federated person index is used in conjunction with a series of business logics that orchestrate data integration from heterogeneous data sources; provide efficient data retrieval; and provide resilient presentation of the data.

Measurement

The feedback system of the present invention comprises a measurement component. This component is constructed through the use of measurement scores and quality indicators. In a preferred embodiment, measurement scores comprise a nationally recognized measure system such as the Health Plan Employer Data and Information Set (HEDIS®) scores. In a preferred embodiment Agency for HealthCare Research and Quality (AHRQ) quality indicators are used to construct the basic measurement system in this invention. These current scores and trends serve as baseline data, and measure anticipated improvements. In one embodiment the scores and trends of Health Maintenance Organizations within a specific community serve as baseline data. In another specific embodiment, the scores and trends of Health Maintenance Organizations in central Massachusetts serve as baseline data.

For example, improvements in the following HEDIS® scores are measured and reported periodically:
Breast Cancer Screening—Percentage of women 50-69 years of age who had a mammogram during the year or the prior year.
Adolescent Immunization—Percentage of adolescents who turned 13 years old who had a second dose of MMR, three hepatitis B and VZV by the 13th birthday.
Colorectal Cancer Screening—Percentage of adults 50-80 years of age who had appropriate screening for colorectal cancer.
Osteoporosis—Percentage of women 67 years of age and older who suffered a fracture, and who had either a BMD (Bone Mineral Density) test or prescription for a drug to treat or prevent osteoporosis in the six months after date of fracture.

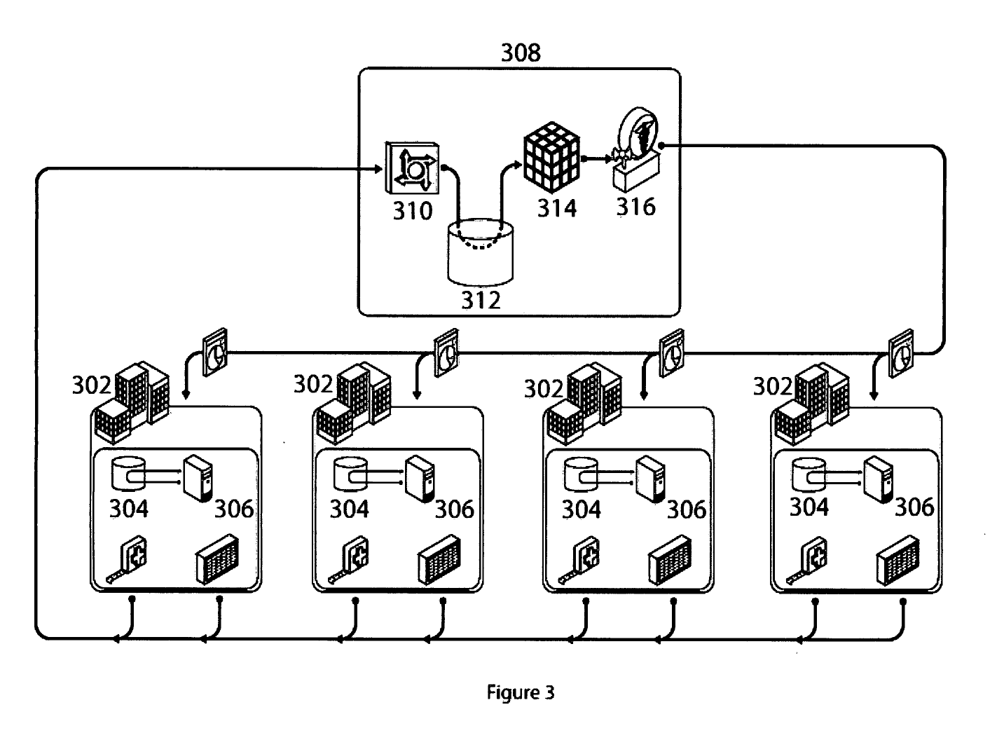

The invention preferably focuses on aggregating and sharing not only clinical data, but also process and outcome data. The computer server(s) include a means by which organizations can submit process/operational and care outcome data to the centralized data repository. For an example of this embodiment, see FIG. 3 and related description set forth below.

In a preferred embodiment, the clinical data and messaging comply with industry standards including but not limited to HL7 and/or SNOMED. Preferably quality care measures include those set forth in Table 1, and patient safety measures include those set forth in Table 2.

As a first example, the following would be determined in a measure set related to diabetes: percentage of patients with diabetes with blood pressure below 140/90 mmHg; percentage of patients with diabetes receiving a dilated eye exam in the past year (or two); percentage of patients with diabetes with low-density lipoprotein (LDL) below 120 mg/dl; percentage of patients with diabetes receiving one or more hemoglobin A1c tests per year; and hemoglobin A1c levels for people with diabetes

As a second example, the following would be determined in a measure set related to a patient's experience of care: time spent with provider; patient's perception of being listened to or having attention given to what he or she says; patient's ability to understand the clinician's explanations; and lead time or wait for appointment and/or care for checkup

It is contemplated that these measure sets will be augmented with new measure sets. For example, in one embodiment, the measure set will be augmented to reflect the regional needs in central Massachusetts. In a second embodiment, the measure sets will be augmented to reflect the perspectives and objectives of participating organizations. Selection criteria as to measure sets will include for example those criteria recommended by national bodies such as the National Healthcare Quality Report Criterions made by the United States Department of Health & Human Services (DHHS), and the HEDIS® criterions made by NCQA (Table 3).

Clinicians will use order forms for missing or overdue tests and procedures that provides awareness of a deficiency that would not otherwise have been noted. The clinician will indicate the test/procedure that needs to be performed and provide that form to the appropriate staff member. The present invention will track such submitted orders to be included in the analysis. By collecting lab data, the present invention further monitors for sentinel events. Sentinel events can include but are not limited to high drug levels (for example digoxin and antiseizure medications), high PT/INR's, rising serum creatinines, and abnormal serum potassium levels.

The present invention also identifies savings realized as a result of the use of the feedback system. To this end varying factors are measured as compared to current baselines and trends. These factors include but are not limited to ER visits, lab/radiology utilization, prescriptions filled, admission rates, inpatient days and the like. The system identifies and reports those admissions that were averted due to information obtained through system use. The financial benefits are calculated and extrapolated to estimate the total savings per user per month.

Time/motion studies in emergency rooms and outpatient clinics, as well as more chart reviews can be conducted to determine how much of a reduction in hospital days was likely due to reduced adverse events versus the reduction in admissions resulting from better information that is made possible via system use.

The infrastructure of the present invention will focus on aggregating and sharing not only clinical data, but also process and outcome data. Built into the system satellite servers and the centralized servers that house utilities, analytical tools and the like that are capable to allow organizations to submit process/operational and care outcome data to the data repositories. See for example in FIG. 3. These data are critical in evaluation the value of healthcare information technology for healthcare industry.

TABLE 1
Components of Health Care Quality and Their Subcategories

| | | Patient | |
|---|---|---|---|
| Safety | Effectiveness | Centeredness | Timeliness |
| 1. Diagnosis | 1. Preventive | 1. Experience | 1. Access to the |
| | care | of care | system of care |
| 2. Treatment | 2. Acute, | 2. Effective | 2. Timeliness in |
| a. Medication | chronic, and | partnership | getting to care |
| b. Follow-up | end-of-life | | for a particular |
| | care | | problem |
| 3. Health | 3. Appropriate- | | 3. Timeliness |
| care environ- | ness of proce- | | within and across |
| ment | dures | | episodes of care |

TABLE 2
Sample Safety Measures from AHRQ

| | |
|---|---|
| Cancer: Screening | Process: % of women (age 40 and over, 50-69) |
| for Breast Cancer | who report they had a mammogram within the |
| | past 2 years |
| | Outcome: % of breast cancers diagnosed at |
| | late stage |
| Cancer: Screening | Process: % of women (18 and over) who report |
| for Cervical Cancer | that they had a Pap smear within the past 3 |
| | years |
| | Outcome: % of cervical cancers diagnosed at |
| | late stage |
| Cancer: Screening | Process: % of men and women (50 and older) |
| for Colorectal | who report they ever had a flexible |
| Cancer | sigmoidoscopy/colonoscopy |
| | Process: % of men and women (50 and over) |
| | who report they had a fecal occult blood |
| | test (FOBT) within the past 2 years |
| | Outcome: % of colorectal cancers diagnosed |
| | at late stage |

TABLE 3
HEDIS ® criterions for selected measures.

| Relevance | Scientific soundness | Feasibility |
|---|---|---|
| Meaningfulness | Clinical evidence | Precise specification |
| Health importance | Reproducibility | Reasonable cost |
| Financial importance | Validity | Confidentiality |
| Cost-effectiveness | Accuracy | Logistical feasibility |
| Strategic importance | Case-mix adjustment | Auditability |
| | or risk adjustment | |
| Controllability | Comparability of data | |
| | sources | |
| Variance among systems | | |
| Potential for improvement | | |

Benchmarking

The community wide clinical data sharing offers additional dimensions and freedoms for the real time regional feedback system to promote uniform patient safety and health care quality. The de-identified information from multiple organizations will be aggregated and analyzed so that regional reports on healthcare quality can be synthesized.

The similar measurement and analytics can be recommended to individual organizations for their internal usage, while the regional reports serve as the benchmark. This way each organization can focus on improving their internal operation for better outcome in terms of quality and safety, referencing their peers and competitors in the region.

Reporting

In a preferred utilization of the present invention the result is a community wide patient safety reporting system and a shared infrastructure to conduct adverse drug event analysis and near miss analysis. The aim of adverse event analysis is to identify ways to improve the delivery of health care through the analysis of adverse events. However, near misses are often precursors of adverse events, and the analysis of their root causes can provide important insights into how to prevent adverse events from happening.

The infrastructure of the present invention provides de-identified adverse drug events and near misses reporting capabilities by member organizations. The definitions of adverse drug events and near misses will be based on industry common practice. The shared system facilitates documentation and review of patient charts, diagnosis, medication, as well as medical-legal documents for adverse drug events and near misses.

Automated surveillance of patient treatment data, including for example clinical patient records, hospital discharge summaries, and Medicare claims data that may be a response to a patient injury are also provided. A set of patterns will be studied and implemented for adverse event detection. Additionally, the progress of patients will be monitored via the disease management channel of the system infrastructure to anticipate conditions that could lead to adverse events and to alert the providers to implement corrective actions. For example, a variety of events can be used as sample triggers for chart reviews and adverse event detection. Event examples include but are not limited to the receipt of diphenhydramine; vitamin K; flumazenil, and the like. The definition and identification of such events are based on industry common practices.

### DETAILED DESCRIPTION OF THE DRAWINGS

FIG. 1 is a schematic illustration of a real time system that polls data from all stakeholders in the regional network. It collects and disseminates these data in the centralized local health information infrastructure (LHII). After the analysis and benchmarking, the reports are sent back to the stakeholders. In practice each participating organization in the system receives local database server(s) 102 and local application server(s) 104. In the illustrated exemplary embodiment, participating organizations are hospital(s) 106, clinic(s) 108, and health plan(s) 110. These local servers collect real time data from the organization. These data are then routed to central LHII 112 where tools such as data warehouse, data analysis/analysis engine, and reporting tools/alert engines are installed. Afterwards the data processing servers process these data and generates the reports. The reports are sent back to each individual organization to view. Alternatively each organization can go to the central portal to view the reports on line.

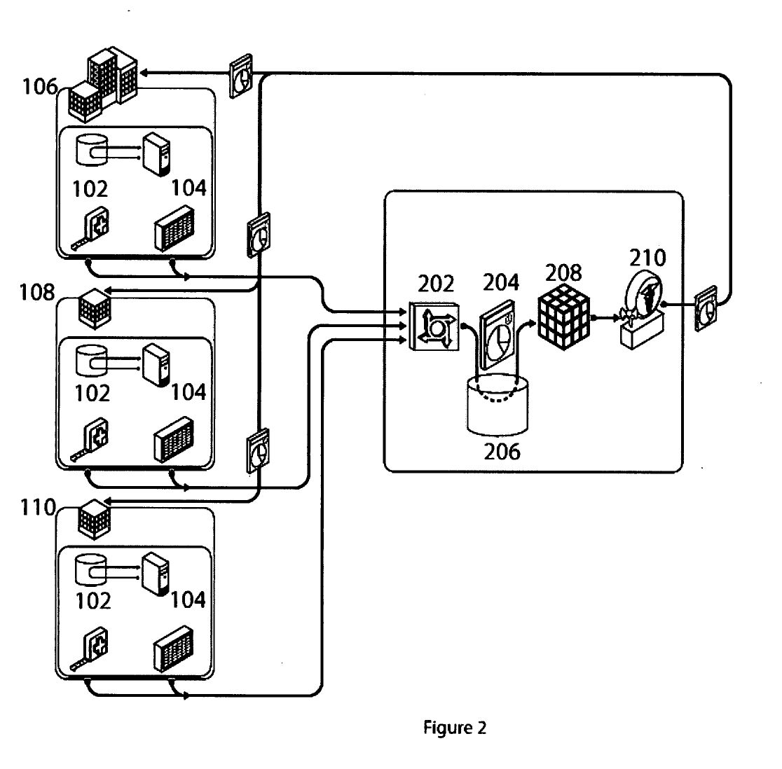

FIG. 2 is a dissection of the real time monitoring system. It builds on service oriented architecture. Each organization in the network routinely funnels real time measurement and monitoring data through Web services 202 to central LHII 204 where the data are stored in data warehouse(s) 206. Analysis is done on the data by way of analysis engine(s) 208 real time and reports and alerts are sent back to the organizations by way of alert engine(s) 210. In practice, each participating organization in the system receives local database server(s) 102 and local application server(s) 104. These local servers collect real time data from the organization. These data are then routed to the centralized data processing servers where the data warehouse, data analysis, and reporting tools are installed. Afterwards the data processing servers process these data and generate the reports. The reports are sent back to each individual organization to view. Alternatively each organization can go to the central portal to view the reports on line.

FIG. 3 is a sample implementation of real time monitoring system for a particular segment of healthcare industry—hospitals. All hospitals in the same region can benefit from participating in such a system where the uniform measures and reports can be very useful for a reference system for improvements in patient safety and care quality. In practice each participating hospital 302 in the system receives local database server(s) 304 and local application server(s) 306. These local servers collect real time data from the hospital. These data are then routed to the central LHII 308 where Web services 310, data warehouse(s) 312, analysis engine(s) 314, and alert engine(s) 316 are installed. Afterwards the data processing servers process these data and generate the reports. The reports are sent back to each individual hospital to view. Alternatively each hospital can go to the central portal to view the reports on line. These reports and measurements can be specific to hospitals while they may be irrelevant to other segments such as payers and employer groups.

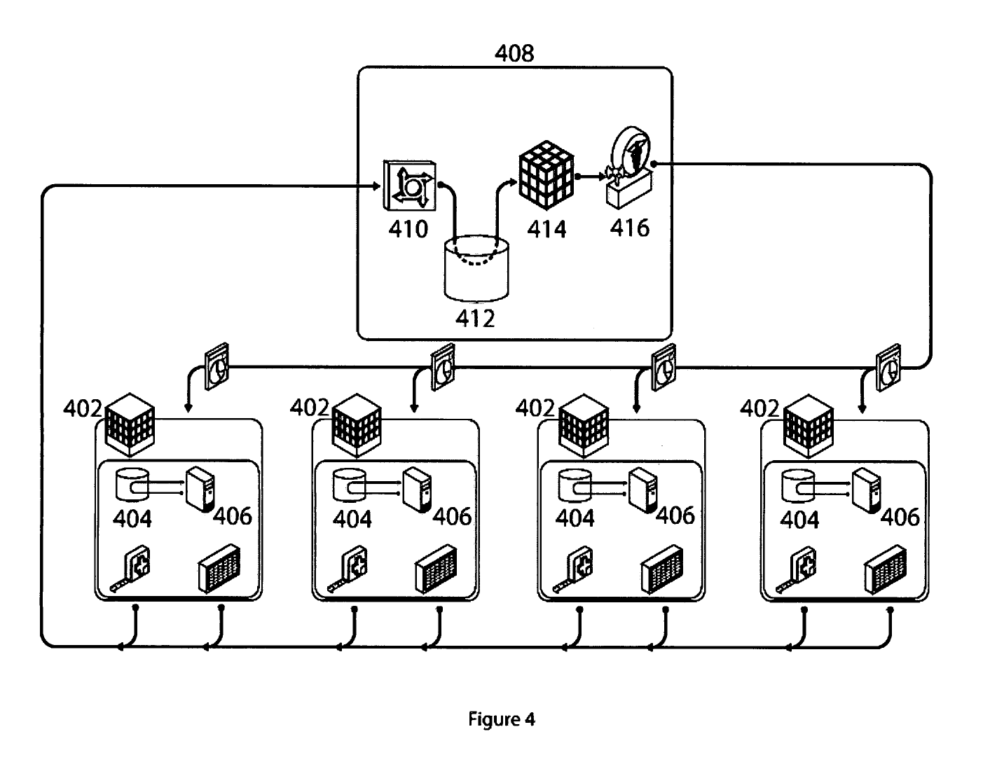

FIG. 4 is a sample implementation of a real time monitoring system for a particular segment of healthcare industry—health plans. In practice, each participating payer or health plan 402 in the system receives local database server(s) 404 and local application server(s) 406. These local servers collect real time data from the payer. These data are routed to central LHII 408 where Web services 410, data warehouse(s) 412, analysis engine(s) 414, and alert engine(s) 416 are installed. Afterwards the data processing servers process these data and generate the reports. The reports are sent back to each individual payer to view. Alternatively each payer can go to the central portal to view the reports on line. These reports and measurements can be specific to payers while they may be irrelevant to other segments such as hospitals and employer groups.

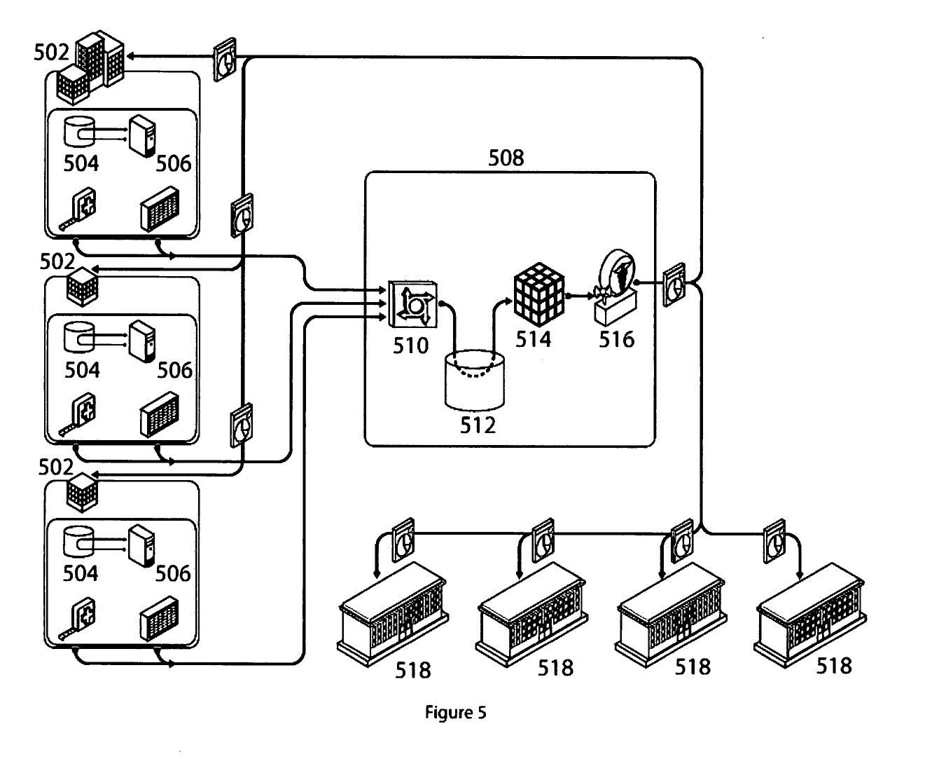

FIG. 5 is an illustration of federated implementation of such real time monitoring system where providers and payers are suppliers of the measurement and monitoring data. The consumer of the analysis and reporting can include but is not limited to government agencies, employers, or research institutions. In practice, each participating organization 502 within the system receives local database server(s) 504 and local application server(s) 506. Such participating organization can include but are not limited to hospital, clinic, or payers. These local servers collect real time data from the local organizations. These data are routed to central LHII 508 where Web services 510 data warehouse(s) 512, analysis engine(s) 514, and alert engine(s) 516 are installed. Afterwards the centralized data processing servers process these data and generate the reports. The reports can be subscribed by government agencies, research institutions, and other stakeholders in the systems. These reports are sent to such subscribers 518. Alternatively each subscriber can go to the central portal to view the reports on line. These reports and measurements can be specific to the subscribers while they may be irrelevant to other stakeholders in the industry.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, since numerous modifications will readily occur to those skilled in the art, it is not desired to limit the invention to the exact construction and operation illustrated and described, and accordingly, all suitable modifications and equivalents may be resorted to, falling within the scope of the invention.

What is claimed is:

1. A monitoring system comprising:
a federated computer network such federated computer network comprising at least one information system, said information system comprising:
a monitoring tool, wherein said monitoring tool monitors data related to a patient visit, and wherein said monitoring tool generates a message, said message comprising information related to said patient visit and
a data processing system, wherein said processing system receives said message, wherein said processing system comprises clinical logics, wherein said processing system applies said clinical logics to said message and determines whether intervention is necessary or whether intervention is not necessary, and when intervention is necessary, said processing system generates and routes at least one alert message;
a data receiver, wherein said data receiver receives said message(s) or said alert message(s) from said information system, said data receiver comprising:
analysis engine(s), said analysis engines comprising at least one of federated master person index; business logic module; or business intelligence module, and wherein said analysis engine analyzes said message(s) and said alert message(s) to create information;
wherein said data receiver compiles and creates reports containing said information.
